(19) United States
(12) Patent Application Publication
(10) Pub. No.: US 2004/0265356 A1
(43) Pub. Date: Dec. 30, 2004

(54) Drug delivery device

(75) Inventor: Linda Mosack, Webster, NY (US)

Correspondence Address: Bausch & Lomb Incorporated, One Bausch & Lomb Place, Rochester, NY, 14604-2701, US

(73) Assignee: Bausch & Lomb Incorporated, Rochester, NY

(21) Appl. No.: 10/610,063

(22) Filed: Jun. 30, 2003

(51) Int. Cl. A61F 2/00

(52) U.S. Cl. 424/428

(57) ABSTRACT

A drug delivery device for placement in the eye includes a drug core comprising a pharmaceutically active agent, and a holder that holds the drug core. The holder is made of a material impermeable to passage of the active agent and includes multiple openings formed by a laser for passage of the pharmaceutically agent therethrough to eye tissue. The number and sizes of the openings are selected so as to obtain a desired release rate of the active agent from the device to the eye.

### FIELD OF THE INVENTION

This invention relates to a drug delivery device, preferably a device that is placed or implanted in the eye to release a pharmaceutically active agent to the eye. The device includes a drug core and a holder for the drug core, wherein the holder is made of a material impermeable to passage of the active agent and includes multiple openings formed by a laser for passage of the pharmaceutically agent therethrough to eye tissue. The number and sizes of openings are selected so as to obtain a desired release rate of the active agent from the device to the eye.

### BACKGROUND OF THE INVENTION

Various drugs have been developed to assist in the treatment of a wide variety of ailments and diseases. However, in many instances, such drugs cannot be effectively administered orally or intravenously without the risk of detrimental side effects. Additionally, it is often desired to administer a drug locally, i.e., to the area of the body requiring treatment. Further, it may be desired to administer a drug locally in a sustained release manner, so that relatively small doses of the drug are exposed to the area of the body requiring treatment over an extended period of time.

Accordingly, various sustained release drug delivery devices have been proposed for placing in the eye and treating various eye diseases. Examples are found in the following patents, the disclosures of which are incorporated herein by reference: U.S. 2002/0086051A1 (Viscasillas); U.S. 2002/0106395A1 (Brubaker); U.S. 2002/0110591A1 (Brubaker et al.); U.S. 2002/0110592A1 (Brubaker et al.); U.S. 2002/0110635A1 (Brubaker et al.); U.S. Pat. No. 5,378,475 (Smith et al.); U.S. Pat. No. 5,773,019 (Ashton et al.); U.S. Pat. No. 5,902,598 (Chen et al.); U.S. Pat. No. 6,001,386 (Ashton et al.); U.S. Pat. No. 6,217,895 (Guo et al.); and U.S. Pat. No. 6,375,972 (Guo et al.).

Many of these devices include an inner drug core including a pharmaceutically active agent, and some type of holder for the drug core made of an impermeable material such as silicone or other hydrophobic materials. The holder includes one or more openings for passage of the pharmaceutically agent therethrough to eye tissue.

A conventional process for forming openings in the drug holder involves manually cutting the openings. However, this can result in excess material around the cut opening from flash or material not being fully removed. Such variations can result in different dosages released through the openings.

### BRIEF DESCRIPTION OF THE DRAWINGS

FIG. 1 is a perspective view of a first embodiment of a drug delivery device of this invention.

FIG. 2 is a cross-sectional view of the device of FIG. 1.

FIG. 3 is a cross-sectional view of a second embodiment of a drug delivery device.

FIG. 4 is a cross-sectional view of a third embodiment of a drug delivery device.

### SUMMARY OF THE INVENTION

According to a first embodiment, this invention relates to a drug delivery device for placement in the eye, comprising: a drug core comprising a pharmaceutically active agent; and a holder that holds the drug core, the holder being made of a material impermeable to passage of the active agent and including multiple openings formed by a laser for passage of the pharmaceutically agent therethrough to eye tissue, wherein the number and sizes of openings are selected so as to obtain a desired release rate of the active agent from the device to the eye.

The invention further provides a method of making a drug delivery device for attachment to eye tissue, comprising: forming openings in a wall of a holder with a laser; and inserting in the holder a drug core comprising a pharmaceutically active agent; wherein the holder is made of a material impermeable to passage of the active agent and the opening permits passage of the pharmaceutically agent therethrough to the eye tissue, and the number and sizes of openings are selected so as to obtain a desired release rate of the active agent from the device to the eye.

According to another embodiment, this invention relates to a method comprising: forming openings in a wall of a holder with a laser; inserting in the holder a drug core comprising a pharmaceutically active agent wherein the holder is made of a material impermeable to passage of the active agent and the openings permit passage of the pharmaceutically agent therethrough; and placing the device in the eye, wherein the number and sizes of openings are selected so as to obtain a desired release rate of the active agent from the device to the eye.

This invention recognized that use of a laser to form the openings in a drug holder permits more precisely formed openings, and allows for selecting the number and sizes of openings to obtain a desired release rate of the active agent from the device to the eye. Additionally, this invention provides methods of making such devices which can be more easily and reliably reproduced on a commercial manufacturing scale.

### DETAILED DESCRIPTION OF PREFERRED EMBODIMENTS

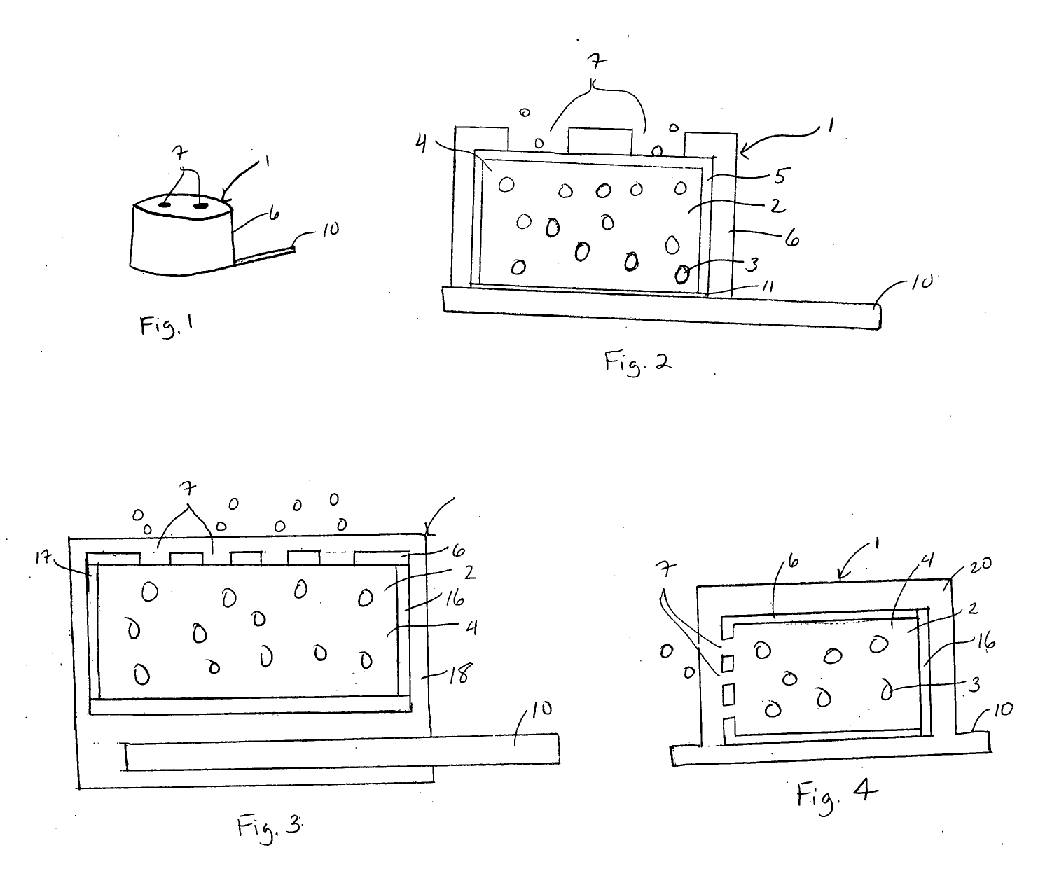

FIGS. 1 and 2 illustrate a first embodiment of a device of this invention. Device 1 is a sustained release drug delivery device for implanting in the eye. Device 1 includes inner drug core 2 including a pharmaceutically active agent 3.

This active agent may include any compound, composition of matter, or mixture thereof that can be delivered from the device to produce a beneficial and useful result to the eye, especially an agent effective in obtaining a desired local or systemic physiological or pharmacological effect. Examples of such agents include: anesthetics and pain killing agents such as lidocaine and related compounds and benzodiazepam and related compounds; anti-cancer agents such as 5-fluorouracil, adriamycin and related compounds; anti-fungal agents such as fluconazole and related compounds; anti-viral agents such as trisodium phosphomonoformate, trifluorothymidine, acyclovir, ganciclovir, DDI and AZT; cell transport/mobility impending agents such as colchicine, vincristine, cytochalasin B and related compounds; antiglaucoma drugs such as beta-blockers: timolol, betaxolol, atenalol, etc; antihypertensives; decongestants such as phenylephrine, naphazoline, and tetrahydrazoline; immunological response modifiers such as muramyl dipeptide and related compounds; peptides and proteins such as cyclosporin, insulin, growth hormones, insulin related growth factor, heat shock proteins and related compounds; steroidal compounds such as dexamethasone, prednisolone and related compounds; low solubility steroids such as fluocinolone acetonide and related compounds; carbonic anhydrize inhibitors; diagnostic agents; antiapoptosis agents; gene therapy agents; sequestering agents; reductants such as glutathione; antipermeability agents; antisense compounds; antiproliferative agents; antibody conjugates; antidepressants; bloodflow enhancers; antiasthmatic drugs; antiparasiticagents; non-steroidal anti inflammatory agents such as ibuprofen; nutrients and vitamins: enzyme inhibitors: antioxidants; anticataract drugs; aldose reductase inhibitors; cytoprotectants; cytokines, cytokine inhibitors. and cytokin protectants; uv blockers; mast cell stabilizers; and anti neovascular agents such as antiangiogenic agents like matrix metalloprotease inhibitors.

Examples of such agents also include: neuroprotectants such as nimodipine and related compounds; antibiotics such as tetracycline, chlortetracycline, bacitracin, neomycin, polymyxin, gramicidin, oxytetracycline, chloramphenicol, gentamycin, and erythromycin; antiinfectives; antibacterials such as sulfonamides, sulfacetamide, sulfamethizole, sulfisoxazole; nitrofurazone, and sodium propionate; antiallergenics such as antazoline, methapyriline, chlorpheniramine, pyrilamine and prophenpyridamine; anti-inflammatories such as hydrocortisone, hydrocortisone acetate, dexamethasone 21-phosphate, fluocinolone, medrysone, methyiprednisolone, prednisolone 21-phosphate, prednisolone acetate, fluoromethalone, betamethasone and triminolone; miotics and anti-cholinesterase such as pilocarpine, eserine salicylate, carbachol, di-isopropyl fluorophosphate, phospholine iodine, and demecarium bromide; mydriatics such as atropine sulfate, cyclopentolate, homatropine, scopolamine, tropicamide, eucatropine, and hydroxyamphetamine; svmpathomimetics such as epinephrine; and prodrugs such as those described in Design of Prodrugs, edited by Hans Bundgaard, Elsevier Scientific Publishing Co., Amsterdam, 1985. In addition to the above agents, other agents suitable for treating, managing, or diagnosing conditions in a mammalian organism may be placed in the inner core and administered using the sustained release drug delivery devices of the current invention. Once again, reference may be made to any standard pharmaceutical textbook such as Remington's Pharmaceutical Sciences for the identity of other agents.

Any pharmaceutically acceptable form of such a compound may be employed in the practice of the present invention, i.e., the free base or a pharmaceutically acceptable salt or ester thereof. Pharmaceutically acceptable salts, for instance, include sulfate, lactate, acetate, stearate, hydrochloride, tartrate, maleate and the like.

For the illustrated embodiment, the active agent employed is fluocininolone acetonide.

As shown in the illustrated embodiment, active agent 3 may be mixed with a matrix material 4. Preferably, matrix material 4 is a polymeric material that is compatible with body fluids and the eye. Additionally, matrix material should be permeable to passage of the active agent 3 therethrough, particularly when the device is exposed to body fluids. For the illustrated embodiment, the matrix material is PVA. Also, in this embodiment, inner drug core 2 may be coated with a coating 5 of additional matrix material which may be the same or different from material 4 mixed with the active agent. For the illustrated embodiment, the coating 5 employed is also PVA.

Device 1 includes a holder 6 for the inner drug core 2. Holder 6 is made of a material that is impermeable to passage of the active agent 3 therethrough. Since holder 6 is made of the impermeable material, passageways 7 are formed in holder 6 to permit active agent 3 to pass therethrough and contact eye tissue. In other words, active agent passes through any permeable matrix material 4 and permeable coating 5, and exits the device through passageway 7. For the illustrated embodiment, the holder is made of silicone, especially polydimethylsiloxane (PDMS) material.

A wide variety of materials may be used to construct the devices of the present invention. The only requirements are that they are inert; non-immunogenic and of the desired permeability. Materials that may be suitable for fabricating the device include naturally occurring or synthetic materials that are biologically compatible with body fluids and body tissues, and essentially insoluble in the body fluids with which the material will come in contact. The use of rapidly dissolving materials or materials highly soluble in body fluids are to be avoided since dissolution of the wall would affect the constancy of the drug release, as well as the capability of the device to remain in place for a prolonged period of time.

Naturally occurring or synthetic materials that are biologically compatible with body fluids and eye tissues and essentially insoluble in body fluids which the material will come in contact include, but are not limited to, glass, metal, ceramics, polyvinyl acetate, cross-linked polyvinyl alcohol, cross-linked polyvinyl butyrate, ethylene ethylacrylate copolymer, polyethyl hexylacrylate, polyvinyl chloride, polyvinyl acetals, plasiticized ethylene vinylacetate copolymer, polyvinyl alcohol, polyvinyl acetate, ethylene vinylchloride copolymer, polyvinyl esters, polyvinylbutyrate, polyvinylformal, polyamides, polymethylmethacrylate, polybutylmethacrylate, plasticized polyvinyl chloride, plasticized nylon, plasticized soft nylon, plasticized polyethylene terephthalate, natural rubber, polyisoprene, polyisobutylene, polybutadiene, polyethylene, polytetrafluoroethylene, polyvinylidene chloride, polyacrylonitrile, cross-linked polyvinylpyrrolidone, polytrifluorochloroethylene, chlorinated polyethylene, poly(1,4′-isopropylidene diphenylene carbonate), vinylidene chloride, acrylonitrile copolymer, vinyl chloride-diethyl fumerale copolymer, butadiene/styrene copolymers, silicone rubbers, especially the medical grade polydimethylsiloxanes, ethylene-propylene rubber, silicone-carbonate copolymers, vinylidene chloride-vinyl chloride copolymer, vinyl chloride-acrylonitrile copolymer and vinylidene chloride-acrylonitride copolymer.

The illustrated embodiment includes a tab 10 which may be made of a wide variety of materials, including those mentioned above for the matrix material and/or the holder. Tab 10 may be provided in order to attach the device to a desired location in the eye, for example, by suturing. For the illustrated embodiment, tab 10 is made of PVA and is adhered to the inner drug core 2 with adhesive 11. Adhesive 11 may be a curable silicone adhesive, a curable PVA solution, or the like.

According to this invention, the openings in holder 6 are formed with a laser, so as to provide uniformly sized, and accurately spaced, cleanly cut holes. Lasers are capable of cutting precision holes of different sizes through polymeric materials accurately and reproducibly, and are capable of being automated. The laser frequency and wavelength may be selected to produce openings with the desired dimensions. Depending on the choice of laser, power and type of material being perforated, the cutting process is either a thermal event, whereby the material is, in essence, melted away, or a nonthermal event, whereby the material is ablated away by breaking chemical bonds in the material.

The process produces holes on the order of 1 to 10 microns. Larger holes on the order of millimeters are produced by programming the laser to sweep across the given dimensions. Lasers such as CO₂ of Nd:YAG are used. Processes such as frequency doubling or tripling YAG lasers are used to produce holes with smaller dimensions. These processes may be automated by placing arrays of holders 6 of devices 1 on a stage and either fixing the position of the devices and sweeping the laser over the holders, or fixing the laser position and moving the holders under the laser to the given coordinates for the openings.

According to preferred embodiments, the holder is also extracted to remove residual materials therefrom. For example, in the case of silicone, the holder may include lower molecular weight materials such as unreacted monomeric material and oligomers. It is believed that the presence of such residual materials may also deleteriously affect adherence of the holder surfaces. The holder may be extracted by placing the holder in an extraction solvent, optionally with agitation. Representative solvents are polar solvents such as isopropanol, heptane, hexane, toluene, tetrahydrofuran (THF), chloroform, supercritical carbon dioxide, and the like, including mixtures thereof. After extraction, the solvent is preferably removed from the holder, such as by evaporation in a nitrogen box, a laminar flow hood or a vacuum oven.

When extraction is used, it is preferably performed prior to the formation of holes in the holder with the laser. This sequence, of extraction followed by formation of the holes, avoids the potential for the extraction process altering the dimensions of the holes. In contrast, in prior processes, where holes were formed with a punch tool, it was generally advisable to perform extraction after forming holes so that the extraction process would remove from the device potential contaminants from the punch tool, but thereby creating the potential for the hole dimensions to be altered by the extraction process.

If desired, the holder may be plasma treated, following extraction, in order to increase the wettability of the holder and improve adherence of the drug core and/or the tab to the holder. Such plasma treatment employs an oxidation plasma in an atmosphere composed of an oxidizing media such as oxygen or nitrogen containing compounds: ammonia, an aminoalkane, air, water, peroxide, oxygen gas, methanol, acetone, alkylamines, and the like, or appropriate mixtures thereof including inert gases such as argon. Examples of mixed media include oxygen/argon or hydrogen/methanol. Typically, the plasma treatment is conducted in a closed chamber at an electric discharge frequency of 13.56 Mhz, preferably between about 20 to 500 watts at a pressure of about 0.1 to 1.0 torr, preferably for about 10 seconds to about 10 minutes or more, more preferably about 1 to 10 minutes.

A device of the type shown in FIGS. 1 and 2 may be manufactured as follows. First, fluocininolone acetonide, the active agent, is provided in the form of a micronized powder, and then is mixed with an aqueous solution of the matrix material, PVA, whereby the fluocininolone acetonide and PVA agglomerate into larger sized particles. The resulting mixture is then dried to remove some of the moisture, and then milled and sieved to reduce the particle size so that the mixture is more flowable. Optionally, a small amount of inert lubricant, for example, magnesium stearate, may be added to assist in tablet making. This mixture is then formed into a tablet using standard tablet making apparatus.

A cylindrical cup of silicone is separately formed, for example by molding, having a size generally corresponding to the tablet and a shape as generally shown in FIG. 2. This silicone holder is then extracted with a solvent such as isopropanol. Openings 7 are placed in silicone with the laser. If desired, a drop of liquid PVA may be placed into the holder through the open end 13 of the holder. Then, the inner drug core tablet is placed into the silicone holder through the same open end and pressed into the cylindrical holder. If the drop of liquid PVA has been applied, the pressing of the tablet causes the liquid PVA to fill the space between the tablet inner core and the silicone holder, thus forming permeable layer 5 shown in FIGS. 1 and 2. A layer of adhesive is applied to the open side of the holder to fully enclose the inner drug core tablet at this end. Tab 10 is inserted at this end of the device. The liquid PVA and adhesive are cured by heating the assembly.

A further benefit of laser cutting of the openings is that the laser process tends to oxidize the silicone holder in the vicinity of the laser cut openings, rendering the surfaces at the holes more hydrophilic and more wettable for later addition of a hydrophilic material such as PVA.

FIG. 3 illustrates another embodiment. In this embodiment, inner drug core 2 may have the form of a tablet, similar to the previous embodiments, including a mixture of active agent 3 and a permeable matrix material 4 such as PVA. Holder 6 may is made of an impermeable material, such as silicone, and in this embodiment, has the form of a tube with impermeable inserts 16, 17 at the ends of the tube. The openings in holder 6 form the passageways 7 for passage of the active agent outside the device. Tab 10 may be made of PVA, and is attached to holder 6 with a permeable coating 18, made of a material such as PVA. Preferably, holder 6 is extracted prior to formation of holes with the laser.

FIG. 4 illustrates another embodiment of this invention. In this embodiment, inner drug core 2 may have the form of a tablet, similar to the previous embodiments, including a mixture of active agent 3 and a permeable matrix material 4 such as PVA. Holder 6 may is made of an impermeable material, such as silicone, and in this embodiment, has the form of a tube with an impermeable insert 16 added after the inner drug core tablet is placed in the holder. The openings in holder 6 form the passageways 7 for passage of the active agent outside the device. In this embodiment, tab 10 is integrally formed, for example by molding, with outer permeable layer 20. Tab 10 may be made of PVA, and in this embodiment, tab 10 circumferentially surrounds the entire device. In this embodiment, holder 6 is preferably extracted prior to formation of holes with the laser.

In each of the aforementioned embodiments, the number and sizes of the holes are selected to obtain a desired release rate of the active agent from the device to the eye. The use of a laser to form the openings in the drug holder permits more precisely formed openings, and allows for more accurately obtained the desired release rate.

It will be appreciated the dimensions of the device can vary with the size of the device, the size of the inner drug core, and the holder that surrounds the core or reservoir. The physical size of the device should be selected so that it does not interfere with physiological functions at the implantation site of the mammalian organism. The targeted disease state, type of mammalian organism, location of administration, and agents or agent administered are among the factors which would effect the desired size of the sustained release drug delivery device. However, because the device is intended for placement in the eye, the device is relatively small in size. Generally, it is preferred that the device, excluding the suture tab, has a maximum height, width and length each no greater than 10 mm, more preferably no greater than 5 mm, and most preferably no greater than 3 mm.

The examples and illustrated embodiments demonstrate some of the sustained release drug delivery device designs for the present invention. However, it is to be understood that these examples are for illustrative purposes only and do not purport to be wholly definitive as to the conditions and scope. While the invention has been described in connection with various preferred embodiments, numerous variations will be apparent to a person of ordinary skill in the art given the present description, without departing from the spirit of the invention and the scope of the appended claims.

What is claimed:

1. A drug delivery device for placement in the eye, comprising:
a drug core comprising a pharmaceutically active agent; and
a holder that holds the drug core, the holder being made of a material impermeable to passage of the active agent and including multiple openings formed by a laser for passage of the pharmaceutically agent therethrough to eye tissue,
wherein the number and sizes of openings are selected so as to obtain a desired release rate of the active agent from the device to the eye.

2. The device of claim 1, wherein the impermeable material comprises silicone.

3. The device of claim 1, wherein a tab is adhered to at least one of the drug core and the holder.

4. The device of claim 1, wherein a tab molded integrally with the holder.

5. The device of claim 1, wherein the drug core comprises a mixture of the active agent and a matrix material permeable to said active agent.

6. The device of claim 5, wherein the matrix material comprises polyvinyl alcohol.

7. The device of claim 1, wherein the holder comprises a cylinder that surrounds the drug core, and an end of the cylinder includes the openings.

8. The device of claim 1, wherein the drug core is cylindrical.

9. The device of claim 1, wherein the drug core is coated with a material permeable to said active agent.

10. The device of claim 1, comprising a mixture of pharmaceutically active agents.

11. A method of making a drug delivery device for attachment to eye tissue, comprising:
forming openings in a wall of a holder with a laser; and
inserting in the holder a drug core comprising a pharmaceutically active agent;
wherein the holder is made of a material impermeable to passage of the active agent and the opening permits passage of the pharmaceutically agent therethrough to the eye tissue, and the number and sizes of openings are selected so as to obtain a desired release rate of the active agent from the device to the eye.

12. The method of claim 11, wherein the impermeable material comprises silicone.

13. The method of claim 11, wherein a tab is adhered to at least one of the drug core and the holder.

14. The method of claim 11, wherein a tab molded integrally with the holder.

15. The method of claim 11, wherein the drug core comprises a mixture of the active agent and a matrix material permeable to said active agent.

16. The method of claim 15, wherein the matrix material comprises polyvinyl alcohol.

17. The method of claim 11, wherein the holder comprises a cylinder that surrounds the drug core, and an end of the cylinder includes the openings.

18. The method of claim 11, wherein the drug core is cylindrical.

19. The method of claim 11, wherein the drug core is coated with a material permeable to said active agent.

20. The method of claim 11, comprising a mixture of pharmaceutically active agents.

21. A method comprising:
forming openings in a wall of a holder with a laser;
inserting in the holder a drug core comprising a pharmaceutically active agent wherein the holder is made of a material impermeable to passage of the active agent and the openings permit passage of the pharmaceutically agent therethrough; and
placing the device in the eye,
wherein the number and sizes of openings are selected so as to obtain a desired release rate of the active agent from the device to the eye.

22. The method of claim 21, wherein a tab is attached to at least one of the holder and the drug core, and the tab is attached to eye tissue by suturing.

23. The method of claim 21, wherein the device is implanted at the back of the eye.
